(19) United States
(12) Patent Application Publication
(10) Pub. No.: US 2002/0022251 A1
(43) Pub. Date: Feb. 21, 2002

(54) Proteinaceous antimycotic complex derived from saccharomyces cerevisiae and its application

(76) Inventors: Nelson Bracesco, Montevideo (UY); Francisco Carrau, Montevideo (UY); Elia Nunes, Montevideo (UY); Virgilio Salvo, Montevideo (UY)

Correspondence Address: ABELMAN FRAYNE & SCHWAB, Attorneys at Law, 150 East 42nd Street, New York, NY, 10017, US

(21) Appl. No.: 09/758,591

(22) Filed: Jan. 10, 2001

(30) Foreign Application Priority Data
Dec. 31, 1998 (UY) 025337

(51) Int. Cl. C12P 21/04; A01N 37/18

(52) U.S. Cl. 435/71.1; 435/942; 514/2

(57) ABSTRACT

An antimycotic protein complex is isolated from a strain of Saccharomyces cerevisiae by incubation at 18-24° C. in a liquid nutrient medium at a pH of 4.5, followed by a centrifugation to separate the cells, filtering and ultrafiltering of the supernanticent liquid to about a 500 to 800-fold increase in concentration, and suspending the concentrate in EDTA.

### FIELD OF INVENTION

The invention relates to a protein complex obtained from a native strain of Saccharomyces cerevisiae (Uruguay) that exhibits very significant antimycotic properties at a broad range of pH and temperature. Obtainment procedure and applications are included in this claim.

### BACKGROUND OF THE INVENTION

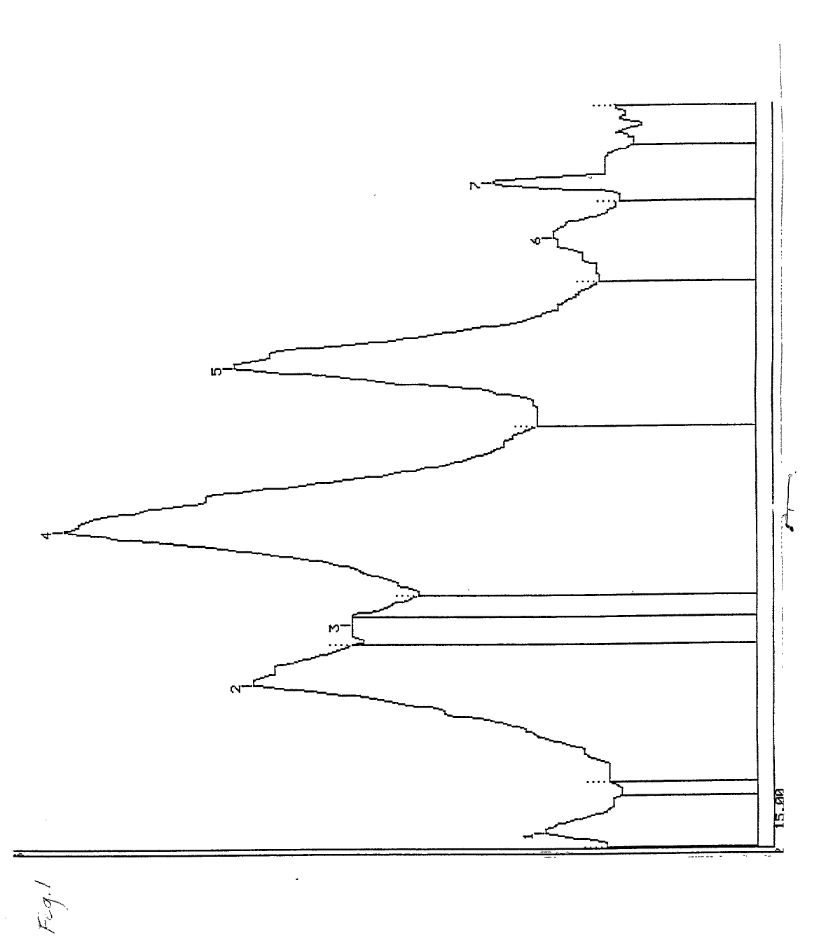

Electrophoresis of the antifungal proteinaceous complex is shown in FIG. 1. This complex is derived from a native killer strain obtained from vineyards of Uruguay. The obtention procedure which is also matter of this invention implies the following steps: culture of the killer yeast strain in filtered culture medium at temperatures between 19 and 24° C., and at a pH value between 4 and 5, filtration and ultrafiltration of the supernatant; 500-800 fold concentration, dialysis, and stabilization in EDTA. This complex of proteins shows stability at a broad range of pH and temperatures including those corresponding to human beings, exhibiting a very significant antimycotic activity. The observed activity at temperatures around 37°. and at pH values around 7, and a corresponding stability at a wide range of temperatures, with a mean life value of at least 6 months at 4° C., differentiate clearly this complex of other products derived from killer strains, that lose their lose activity and stability at temperatures above 30° C. and pH higher than 5 (Palfree and Bussey, 1979; Woods and Bevan 1968; Hodgson et al, 1995). Preliminar tests in rodents (n=12) and in 3 human volunteers indicate lack of toxicity and of secondary effects, opposite to observations of other authors using products derived from other yeast species (Polonelli et al., 1991). Therefore, we claim as invention the application of this complex for the production of antimycotics for medical-, veterinary-, and agriculture-uses in different systems. As an example, the lethal effect on Candida albicans and Tricophyton mentagrophytes, two of the most resistant mycosis at human level are presented. The antifungal effect is observed both in exponentially and in stationary phase of growth. It should be taken into account that the incidence of human mycosis is continuously increasing, due in part to the AIDS pandemia, and to the immune deficiency conditioned by certain cancer treatments. On the other side, a hallmark of mycosis in different living systems is the continuous increasing resistance to different types of treatments.

It is known that certain yeast strains so called “killers” can synthesize in certain conditions proteinaceous toxins that can induce death of other sensitive strains (Mitchel et al., 1973; Mitchel and Bevan, 1983; Wickner, 1996). The killer phenomenon is frequently observed in native strains and has ecological importance, givin growth advantages in different conditions. It is associated with double stranded RNA genomes whish are encapsidated in viral particles. It was demonstrated that the optimal pH value for the induction of corresponding toxins is around 4.5, the production temperature being around 20° C. (Woods and Bevan, 1968). An open literature revision indicates a wide use of similar products specially in the wine industry at a pH value of 3.5-4.0 and lack of activity and stability at pH 7 and temperatures higher than 30° C. (Palfree and Bussey, 1979; Woods and Bevan, 1968; Hodgson et al 1995). A related product obtained from another species of yeast (Pichia anomala) showed activity against Pneumocystis carinii, but it was highly toxic in rodents (Polonelli et al, 1991; Petoello-Montovani et al, 1995). The synthesis of antiidiotypic antibodies of this toxin perfomed by these authors (Magliani et al, 1997), gave a product with significantly lower antifungal activity “in vitro” as compared with the complex presented here. Other authors have isolated by HPLC (high perfomance liquid chromatography) a product secreted by Saccharomyces boulardii grown at 30-37° C. which has antibacterial activity at a pH value of 5.5. (Friedland and Seifert, European Patent Office, 0650731 A 1).

Hodgson et al (1995) performed a toxin activity screening against Candida albicans using different killer species including S. cerevisiae, Pichia and Kluyveromyces showing a relatively high sensitivity for the Willipsis mrakii toxin. However, in 90% of all perfomed test a lethality lower than 66% was observed. Interestingly, in case of Saccharomyces no induced lethality was observed for one of the used strains meanwhile for the other used strain (NCYC 761) the lethality was lesser than that induced by W. mrakii. Similary to previous studies, unstability at temperatures higher than 30° C. and pH higher than 4 was observed. On the other side, the activity of the most active toxins was maintained only during 24 h. There is evidences that the killer toxins secreted by different types of cells determine the formation of ionic channels in the presence of membrane receptors, changing selective permeability and producing cell death (Hodgson et al, 1995, for revision).

The proteinaceous complex derived of the native killer yeast strain (KU1) contains 7 associated proteins of molecular weights between 12 and 67 KD (FIG. 1).

Assuming a 1 to 1 stecheometry for receptors and active toxins we propose that at high concentration of the complex an increase in receptor—ligand affinity (cooperative effect) enables saturation of all vacant membrane receptors in the sensitive cells, inducing 100% lethality provided by the obtained toxin stability in a broad range of pH and T.

In order to obtain the protein complex derived of the KU1 secretion product following procedure was performed.

The KU1 killer strain Saccharomyces cerevisiae was grown in Halvorson media plus YPD (1/1), filtered and ultrafiltered with exclusion for 10 KD, in thermostatized water bath at 20° C. Cells in the exponential phase of growth were centrifuged (500 g, 10 min). The supernatant was filtered, ultrafiltered (pore diameter<10 KD), and concentrated 500-800 fold. Thereafter the concentrate was resuspended in EDTA.

The antimycotic activity of the complex can be expermientaly demonstrated through the following “in vitro” tests.

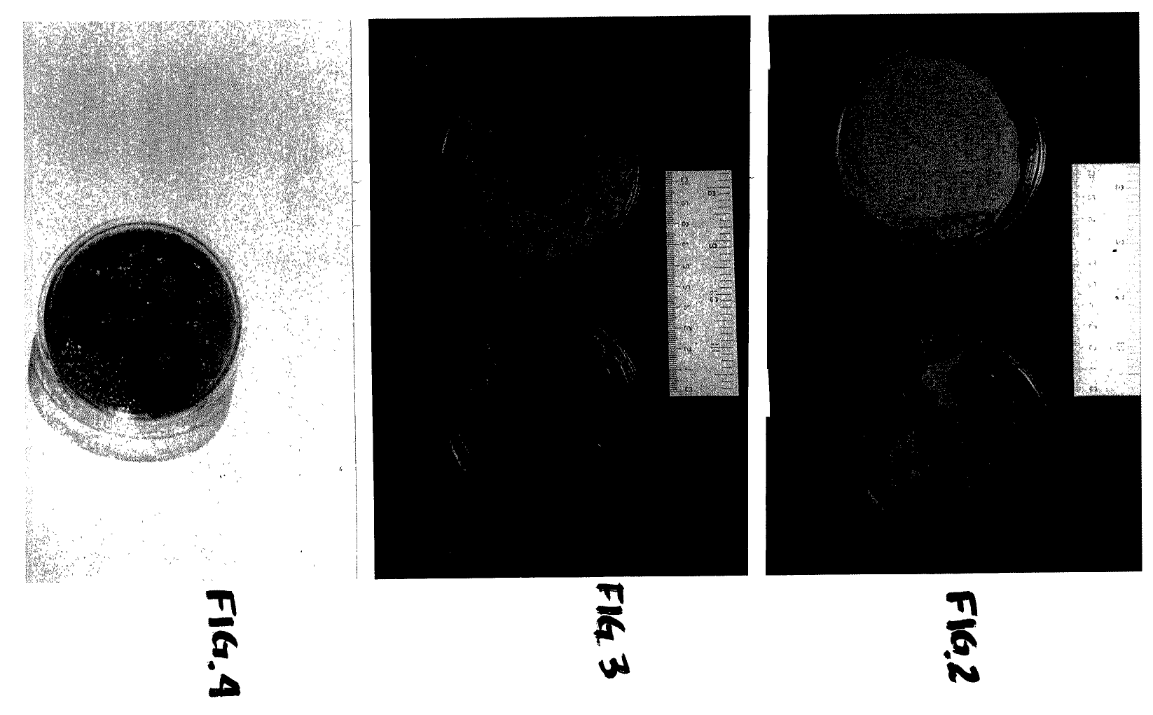

Candida albicans and Tricophyton mentagrophytes cultures grown on Sabouraud medium at 37° C. and pH7 (human physiological condition simulation) were exposed during different times to the complex. In all cases, an inactivation halo (Hershkovitz, 1988), corresponding to dead cells as determined by methylene blue (0.003%) was observed. Correspondig control cultures without exposure showed normal growth in the same conditions. (FIGS. 2+3). The lethal power of the complex was compared with that of ketoconazole, an antimycotic drug which is currently used at the human level. Used in the same conditions as described for FIGS. 2+3 and at a concentration of 200 ug/ml, the effect of the “killer complex” was significantly higher (p<0.001). A comparison of the lethal power of the killer complex with that of the Amphotericin B (an antimycotic drug, toxic at the human level) also showed a significant higher effect regarding survival of sensitive Saccharomyces cerevisiae cells. (p<0.001).

We claim:

1. Product that consists of a proteinaceous complex derived of a killer yeast secretion compound with eletrophoresis diagram shown in FIG. 1. It is obtained by incubation of native uruguayan killer Saccharomyces cerevisiae yeast strains at 18-24° C. in liquid-nutrient medium at pH 4.5, followed by separation of the cells by centrifugation, filtering and ultrafiltering of the supernatant, 500-800 fold concentration, dialysis and suspension in EDTA 12.5 Mm.

2. Product with the characteristics described in item (1) that requires incubation of killer yeast strains at 18-25° C.

3. Product with characteristics described in items (1 and 2) that requires culture of the killer yeast in nutrient YP-Halvorson medium at pH 4.5.

4. Products with characteristics described in items (1-3) that requires separation of the yeast cells by centrifugation.

5. Product with characteristics described in items (1-4) that requires filtering, ultrafiltering and concentration of the cell free supernatant by a factor of 500-800.

6. Application of the product with the described characteristics (1-6) for medical, veterinary- and agriculture-uses.
